(19) United States
(12) Patent Application Publication
(10) Pub. No.: US 2024/0238102 A1
(43) Pub. Date: Jul. 18, 2024

(54) TIBIAL SAMPLE IMPLANT WITH GEARING MECHANISM

(71) Applicant: Aesculap AG, Tuttlingen (DE)

(72) Inventors: Martin Nonnenmann, Wurmlingen (DE); Jennifer Mader-Ilg, Seitingen-Oberflacht (DE); Housseyn Zouaghi, Chaumont (FR)

(21) Appl. No.: 18/014,790

(22) PCT Filed: Jul. 5, 2021
(86) PCT No.: PCT/EP2021/068515

§ 371 Date: Jan. 6, 2023

(30) Foreign Application Priority Data
Jul. 7, 2020 (DE) 10 2020 208 501.3

(51) Int. Cl. A61F 2/46; A61F 2/30; A61F 2/38

(52) U.S. Cl. CPC A61F 2/4684; A61F 2/389; A61F 2/461; A61F 2002/30354; A61F 2002/30398; A61F 2002/30405; A61F 2002/30482; A61F 2002/30525; A61F 2002/30553; A61F 2002/30556; A61F 2002/30617; A61F 2002/3085; A61F 2002/4628; A61F 2002/4661

(57) ABSTRACT

A tibial sample implant for use in knee joint replacement surgery has a lower part and an upper part. The upper part has a sliding surface designed to interact with a femoral component. A height adjustment mechanism can move the upper part in a guided manner relative to the lower part between a first position, in which the sliding surface is positioned at a first height above the lower part, and a second position, in which the sliding surface is positioned at a second height above the lower part. The height adjustment mechanism has a drive gear and a control cam. An output element is rotationally fixed to the upper part and has a support section supported on the control cam so that the upper part can be moved between the first position and the second position by a rotational movement of the drive gear.

### CROSS-REFERENCE TO RELATED APPLICATIONS

This application is the United States national stage entry of International Application No. PCT/EP2021/068515, filed Jul. 5, 2021, and claims priority to German Application No. 10 2020 208 501.3, filed Jul. 7, 2020. The contents of International Application No. PCT/EP2021/068515 and German Application No. 10 2020 208 501.3 are incorporated by reference herein in their entireties.

### FIELD

The invention relates to a tibial trial implant for use in a knee joint replacement operation, comprising a lower part, which is provided for tibial fixation, an upper part, which is arranged above the lower part in the height direction and has a glide face that is arranged on the upper side and is provided for gliding interaction with a femoral component, and comprising a height adjustment mechanism which is actively connected to the upper part and the lower part and by means of which the upper part can be guided displaceably relative to the lower part in the height direction between a first adjustment position, in which the glide face is positioned at a first height above the lower part, and a second adjustment position, in which the glide face is positioned at a second height above the lower part.

### BACKGROUND

Such a tibial trial implant is known from US 2010/0063595 A1, and comprises a lower plate and an upper plate. A glide face, which is intended for gliding interaction with a femoral component, is provided on an upper side of the upper plate. The lower side of the lower plate is provided for tibial fixation. The tibial trial implant furthermore comprises a height adjustment mechanism, which is arranged between the upper plate and the lower plate and is actively connected to the two plates. The height adjustment mechanism allows adjustment of the overall height of the trial implant. For this purpose, the upper plate and the lower plate are displaced relative to one another in the height direction by means of the height adjustment mechanism. In the known tibial trial implant, the height adjustment mechanism comprises a shear joint arrangement which is connected at one end firmly to the lower plate and at the other end firmly to the upper plate. For manual actuation of the height adjustment mechanism, a tilting lever secured so as to move pivotably on the lower plate is provided.

### SUMMARY

It is an object of the invention to provide a tibial trial implant of the type mentioned in the introduction, which has an improved structure in comparison with the prior art and offers advantages during use.

This object is achieved in that the height adjustment mechanism comprises a cam gear having at least one drive wheel, which is mounted rotatably on the lower part about a first rotation axis oriented parallel to the height direction and comprises a control cam rising in the height direction, and having at least one driven element, which is firmly connected to the upper part and comprises a supporting section glidingly supported on the control cam, so that the upper part can be displaced by means of a rotational movement of the drive wheel between the first adjustment position and the second adjustment position. The cam gear provided according to the invention allows particularly advantageous adjustment of the height of the glide face and a particularly advantageous structure of the tibial trial implant. This is because the drive-side rotational movement of the drive wheel can be transmitted in a straightforward and robust way via the interaction of the control cam with the supporting section into a driven-side movement of upper part and of the glide face arranged thereon. By a corresponding configuration of the control cam, very different proportional or non-proportional transmission ratios may be produced between the driving-side movement and the driven-side movement with low design complexity. Furthermore, in particular because of the rotation axis of the drive wheel oriented parallel to the height direction, a particularly compact construction of the tibial trial implant may be achieved. As a result, a simple, space-saving and at the same time robust structure is achieved by the solution according to the invention. The drive wheel is configured for at least indirect manual and/or tool-driven actuation by medical personnel. For example, the drive wheel may comprise an actuation section provided for this purpose on its outer circumference. Alternatively or in addition, an actuation element configured for manual and/or tool-driven actuation may be provided, which precedes the drive wheel and interacts therewith so that a movement of the actuation element can be transmitted onto the drive wheel. The drive wheel is preferably mounted glidingly on the lower part. The control cam interacts glidingly with the supporting section of the driven element during a rotation of the drive wheel. The supporting section is supported on the control cam in the height direction. During a rotation of the drive wheel, a relative gliding movement takes place between the control cam and the supporting section. In other words, the control cam glides through under the supporting section. The supporting section is thereby pressed upward in the height direction because of the pitch of the control cam. The control cam and the supporting section may interact pointwise, along a line and/or over a surface. The control cam may have a constant or varying pitch. The driven element is connected firmly to the upper part. Preferably, the driven element is formed integrally onto the upper part.

Preferably, the driven element is arranged on the lower side on the upper part. The glide face is preferably formed on the upper part and constitutes its upper side. Alternatively, the glide face may be formed on a separate part which is connected to the upper side of the upper part. The lower part is provided for the tibial fixation. During use, the lower side of the lower part may be fixed directly on the proximal end of the tibia. Alternatively, the lower part may be fixed on a directly tibially fastened tibial trial plateau or the like. The lower part and the upper part may respectively be configured in one or more pieces. Preferably, the lower part and the upper part respectively have a plate-like basic shape, so that a lower and upper plate may respectively be referred to. In the first adjustment position, the tibial trial implant has an overall height extending in the height direction which is less than an overall height in the second adjustment position. Accordingly, the glide face is positioned closer to the lower part in the height direction in the first adjustment position than in the second adjustment position.

In one embodiment of the invention, the control cam is formed by a control face helically extending with a constant pitch coaxially around the first rotation axis. The control cam therefore describes a screw line rising in the height direction. Because of the constant pitch, a linear movement law between the drive wheel and the driven element, and therefore proportional movement and force transmission, may be achieved in a simply designed way. This contrasts with the shear joint arrangements known from the prior art for height adjustment of the glide face. This embodiment of the invention therefore allows further advantages during use. In addition, the installation space may be reduced further.

In another embodiment of the invention, the supporting section is formed by a supporting face helically extending with a constant pitch coaxially around the first rotation axis. Because of the two-dimensional configuration of the supporting section, in particular a reduced surface pressure may be achieved on the control cam. In this way, the height adjustment mechanism may be operated smoothly and with low wear.

In another embodiment of the invention, the upper part and the lower part are releasably plugged together by means of a plug connection formed between the at least one drive wheel and the at least one driven element while being glidingly mobile relative to one another along a plug axis, the plug axis being oriented coaxially with the first rotation axis. This is a particularly advantageous embodiment of the invention. The cam gear is in this case provided with a particularly advantageous multiple function. This is because in addition to the adjustment of the height of the glide face, the height adjustment mechanism functions as a releasable interface between the upper and lower parts. For this purpose, the plug connection is formed between the drive wheel and the driven element. The plug connection is configured in such a way that the driven element—and therefore also the upper part firmly connected to the driven element—can be retracted upward from the lower part. For this purpose, the plug axis is oriented coaxially with the first rotation axis and therefore parallel to the height direction. This embodiment of the invention makes it possible that the lower part and the components of the height adjustment mechanism which are mounted thereon can be connected straightforwardly to different upper parts, which for example comprise differently configured glide faces. This contrasts with solutions known from the prior art, which provide nonreleasable or at least manually nonreleasable coupling between the lower part and the upper part. The different upper parts may, for example, also differ in their anteroposterior dimension, mediolateral dimension, height and/or, in the case of an asymmetric configuration of the glide face, also by mirror-symmetrical variants for the left and right knee, respectively.

In another embodiment of the invention, the at least one drive wheel comprises a cylindrical bore extending coaxially with the first rotation axis, into which a complementary plug cylinder of the at least one driven element is plugged so as to form the releasable plug connection. The plug cylinder is plugged glidingly movably into the cylindrical bore along the plug axis, and therefore along the height direction. This on the one hand allows simple release of the plug connection. On the other hand, it allows the relative movement between the upper and lower parts which is necessary for adjusting the height of the glide face.

In another embodiment of the invention, the height adjustment mechanism comprises a worm gear preceding the cam gear. “Preceding” means that the worm gear precedes the cam gear in the drive direction. In other words, the cam gear is driven by means of the worm gear. The preceding worm gear allows particularly high transmission ratios in a simply designed and space-saving way. In this way, precise adjustment of the height of the glide face may be achieved. Furthermore, high spreading forces may be produced between the upper and lower parts if required.

In another embodiment of the invention, the worm gear comprises self-retention. “Self-retention” means that the worm gear can be driven only via its gear input. Conversely, driving of the worm gear starting from its gear output is not possible, for example due to friction and/or transmission. In this way, locking of the upper part in the different adjustment positions is obviated. This allows a further simplified design in this regard. Furthermore, a self-retaining configuration of the cam gear for this purpose may be obviated. The cam gear may therefore be configured primarily with a view to a movement law to be achieved between the lower part and the upper part.

In another embodiment of the invention, the worm gear comprises a worm drive shaft, which is mounted rotatably on the lower part about a second rotation axis oriented perpendicularly to the height direction, the worm drive shaft interacting with a toothed outer circumferential section of the at least one drive wheel or with a worm wheel preceding the at least one drive wheel. By the corresponding orientation of the second rotation axis, a compact construction of the tibial trial implant may be achieved in the height direction. The worm drive shaft may act directly or indirectly on the at least one drive wheel. For this purpose, the at least one drive wheel may comprise a toothed section on its outer circumference, namely the toothed outer circumferential section. The teeth of the worm drive shaft engage in the teeth of the outer circumferential section in order to drive the drive wheel. If only indirect driving is provided, the worm drive shaft acts on the worm wheel preceding the drive wheel. This worm wheel is preferably mounted rotatably on the lower part about a third rotation axis, which is oriented parallel to the height direction.

In another embodiment of the invention, the worm drive shaft comprises on one end a rotation actuation section, which is configured for manual and/or tool-driven rotation actuation of the worm drive shaft about the second rotation axis. The rotation actuation section may in particular be configured as a rotary knob, crank or the like for manual actuation. Alternatively or in addition, a tool face for tool-driven rotation actuation may be provided on one end of the rotation actuation section. The rotation actuation section is arranged externally accessibly on the lower part.

In another embodiment of the invention, the at least one drive wheel and/or the worm drive shaft is glidingly mounted rotatably on a glide bearing face of the lower part, the glide bearing face comprising a plurality of radial flushing protrusions. The flushing protrusions allow simple flushing of the glide bearing formed between the drive wheel and/or the worm drive shaft and the lower part. In this way, particularly hygienic use of the tibial trial implant may be achieved.

In another embodiment of the invention, a scale display is formed between the lower part and the at least one drive wheel, which comprises a reading element and a scale and allows reading of the adjustment position of the upper part and/or of the height of the glide face. During a rotational movement of the drive wheel, the reading element and the scale are moved relative to one another. The reading element may be arranged on the lower part and the scale may be arranged on the drive wheel, or vice versa. In another embodiment, a plurality of scale displays are formed in order to allow reading from different viewing angles. Alternatively, in the case of two or more scale displays, the values to be read may be arranged alternately on the individual scales in order to allow good readability despite finely graduated values.

In another embodiment of the invention, the lower part is configured as two shells and comprises an upper shell and a lower shell, the at least one drive wheel and/or the worm drive shaft being held between the upper shell and the lower shell. In an assembled state, the upper shell and the lower shell form a housing for mounting of the at least one drive wheel and/or of the worm drive shaft.

In another embodiment of the invention, the height adjustment mechanism is configured at least substantially, preferably fully, mirror-symmetrically with respect to a vertical mid-length plane, at least two drive wheels and at least two driven elements being provided. The drive wheels and the driven elements are therefore arranged next to one another in the transverse direction. If the cam gear is preceded by a worm gear, its worm drive shaft preferably drives both drive wheels. For this purpose, the worm drive shaft projects between the two drive wheels in the longitudinal direction.

In another embodiment of the invention, there are at least two drive wheels and one control wheel, which respectively comprise spur teeth, the control wheel being spur-meshed with the two drive wheels. The at least two drive wheels are preferably arranged spaced apart from one another in the transverse direction. The control wheel is arranged between the at least two drive wheels in the transverse direction and precedes them. The control wheel is used for force and movement coupling of the two drive wheels. Because of the coupling by means of the control wheel, the two drive wheels are rotationally movable synchronously with one another about their respective rotation axis. Preferably, the control wheel is rotationally movably mounted on the lower part. In one embodiment, the control wheel is configured for direct manual rotation actuation. In another embodiment, the control wheel may be configured to interact with actuation mechanics or the like. The spur teeth are respectively outer teeth. The rotation axes of the at least two drive wheels and of the control wheel are oriented parallel to one another.

In another embodiment of the invention, the at least one control wheel comprises a further control cam, which interacts with a further supporting section of a further driven element. The control wheel therefore functions so to speak as a further drive wheel. In other words, in this embodiment there are at least three drive wheels and three driven elements, one of the drive wheels so to speak being formed by the at least one control wheel. The at least one control wheel is thereby provided with a particularly advantageous multiple function. In this way, a particularly compact and at the same time particularly robust structure is achieved.

In another embodiment of the invention, the at least one drive wheel is coupled so as to transmit movement to at least one display wheel which is rotationally movably mounted on the lower part, the display wheel comprising a scale which allows reading of the adjustment position of the upper part and/or of the height of the glide face. The movement transmission between the drive wheel and the display wheel may be continuous or intermittent. For the movement transmission, the display wheel may be meshed with the drive wheel or coupled in another way, for example by means of a catch element. Preferably, the display wheel is arranged on an outer circumference of the lower part and/or offset outward in the radial direction relative to the at least one drive wheel. The scale preferably comprises a sequence of graduation lines and/or numbers. The scale is preferably arranged on an outer circumference of the display wheel.

In another embodiment of the invention, there is at least one catch element connected fixed in rotation to the drive wheel, which intermittently interacts with the display wheel so that a continuous rotational movement of the drive wheel causes a stepwise rotational movement of the display wheel. The stepwise rotational movement of the display wheel which is caused in this way leads to a stepwise display of the respectively reached adjustment positions of the upper part. Intermediate positions, on the other hand, are not displayed. The catch element may be formed integrally on the drive wheel or configured as a component part which is manufactured separately and subsequently connected to the drive wheel. The catch element interacts intermittently with a section of the display wheel which is intended therefor.

In another embodiment of the invention, there is a latch device which comprises a latch element actively connected fixed in rotation to the at least one drive wheel and a complementary latch counter-element arranged on the lower part, the latch element and the latch counter-element interacting in defined rotational settings of the at least one drive wheel while forming an overridable latch connection. The defined rotational settings of the at least one drive wheel correspond to adjustment positions of the upper part which are predetermined and/or are to be reached. In this way, a user receives direct tactile and/or acoustic feedback as soon as said rotational setting and/or adjustment position is reached. This feedback is given by the latching of the latch device. The latch connection between the latch element and the complementary latch counter-element is overridable, so that a further rotational movement of the drive wheel is also possible after reaching the defined rotational setting. In this case, the override—that is to say the release of the previously established latch connection—is associated with new tactile and/or acoustic feedback for the user.

In another embodiment of the invention, there is a handle releasably connectable to the lower part, having actuation mechanics, the actuation mechanics comprising an actuation wheel which is actively connected to the at least one drive wheel in a state of the handle connected to the lower part. The handle allows ergonomic actuation of the height adjustment mechanism. For this purpose, the actuation mechanics of the handle precede the height adjustment mechanism, or more precisely its at least one drive wheel. Manual actuation of the actuation mechanics causes a rotational movement of the at least one drive wheel and therefore a corresponding change in the adjustment position of the upper part. Furthermore, the handle allows secure and ergonomic handling of the lower part. For this purpose, the handle can be connected releasably to the lower part. Preferably, for this purpose, between the handle and the lower part there is a releasable latch, snap, clamp and/or plug connection which comprises at least one connecting element assigned to the handle and a connecting counter-element complementary thereto on the lower part. The actuation wheel of the actuation mechanics is—in the state of the handle connected to the lower part—actively connected releasably to the at least one drive wheel. Preferably, the actuation wheel and the at least one drive wheel are for this purpose meshed at least indirectly with one another. A transmission ratio between the rotational movement of the actuation wheel and the rotational movement of the at least one drive wheel may be influenced straightforwardly by means of a corresponding configuration of the actuation mechanics. Depending on the selected transmission ratio, particularly precise and smooth or particularly rapid adjustment of the height adjustment mechanism may be achieved.

In another embodiment of the invention, the handle comprises at least one connecting element arranged at its distal end, which is releasably connected to a complementary connecting element of the lower part in order to connect the handle to the lower part. This embodiment allows a particularly simple and robust structure. Preferably, the connecting elements are configured as a latch element and a latch counter-element. Alternatively, a plug or clamp connection with correspondingly configured connecting elements may be envisioned. When establishing and releasing the latch connection, the latch element and/or the complementary latch counter-element are resiliently deformed. Preferably, there are at least two latch elements and complementary latch counter-elements arranged spaced apart from one another in the transverse direction. The handle extends longitudinally between the distal end and its proximal end. Preferably, the actuation wheel of the actuation mechanics is arranged distally.

### BRIEF DESCRIPTIONS OF THE DRAWING FIGURES

Further advantages and features of the invention may be found from the following description of preferred exemplary embodiments of the invention, which are represented with the aid of the drawings.

FIG. 1 shows a perspective view of an embodiment of a tibial trial implant according to the invention,

FIG. 2 shows the tibial trial implant according to FIG. 1 in an exploded perspective view,

FIG. 3 shows a further exploded perspective view,

FIG. 4 shows the tibial trial implant according to FIGS. 1 to 3 in schematic sectional representation along a section A-A according to FIG. 5,

FIG. 5 shows a schematic front view of the tibial trial implant according to FIGS. 1 to 4,

FIG. 6 shows a further perspective view with graphical omission of individual components and/or sections of the tibial trial implant,

FIG. 7 shows a perspective detail view of two drive wheels of a height adjustment mechanism of the tibial trial implant according to FIGS. 1 to 6,

FIG. 8 shows a further perspective view similar to the representation according to FIG. 6 in a viewing direction directed obliquely upward,

FIG. 9 shows a view corresponding to FIG. 8 with additional graphical omission of the drive wheels,

FIG. 10 shows a perspective view of a further embodiment of a tibial trial implant according to the invention together with a tibial trial plateau and in a first adjustment position,

FIG. 11 shows the tibial trial implant according to FIG. 10 in a second adjustment position,

FIG. 12 shows an exploded perspective view of the tibial trial implant according to FIGS. 10 and 11,

FIG. 13 shows a perspective view of an upper part of the tibial trial implant according to FIGS. 10 to 12 with a viewing direction onto a lower side, on which driven elements of a height adjustment mechanism are arranged,

FIG. 14 shows a perspective view of a lower part of the tibial trial implant according to FIGS. 10 to 12,

FIG. 15 shows a further perspective representation of the lower part in a partially exploded view,

FIG. 16 shows a further view of the lower part with a viewing direction onto individual components and/or sections of the height adjustment mechanism,

FIG. 17 shows a perspective view of a handle of the tibial trial implant according to FIGS. 10 to 12, which can be connected releasably to its lower part and comprises actuation mechanics for actuating the height adjustment mechanism, and

FIG. 18 shows a perspective detail representation of a distal end of the handle according to FIG. 17.

### DETAILED DESCRIPTION

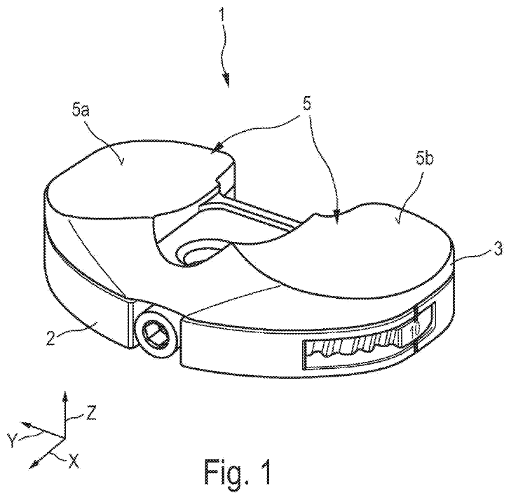

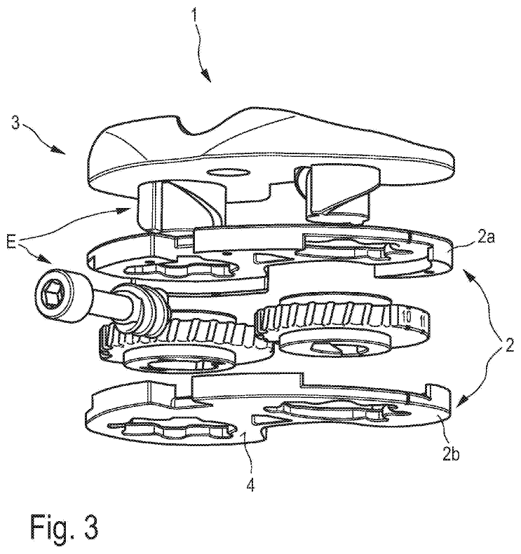

According to FIGS. 1 to 3, a tibial trial implant 1 for use in a knee joint replacement operation is provided. The tibial trial implant 1 is often referred to in medical terminology as a “trial glide face”.

The tibial trial implant 1 comprises a lower part 2, an upper part 3 and a height adjustment mechanism E.

The lower part 2 is provided for the tibial fixation. This means that when the tibial trial implant 1 is being used, the lower part 2 is arranged and fastened indirectly or directly on a proximal end of a tibia. In the embodiment shown, indirect fixation of the lower part 2 is provided, in which a lower side 4 of the lower part 2 interacts with a so-called tibial trial plateau, which may be screwed to or cemented into the proximal end of the tibia.

In the embodiment shown, the lower part 2 comprises a plate-like basic shape. Accordingly, the lower part 2 respectively extends further along a longitudinal direction X and a transverse direction Y compared with in a height direction Z.

In the present case, the lower part 2 has a two-shell configuration with an upper shell 2a and a lower shell 2b, which is advantageous in a manner described in more detail below but is not absolutely necessary. The lower shell 2b is arranged below the upper shell 2a in relation to the height direction Z. The lower shell 2b comprises the lower side 4. In an assembled state, the upper shell 2a and the lower shell 2b form a reception space (not denoted in detail) for component parts and/or sections described in more detail below, in particular of the height adjustment mechanism E. In the mounted state ready for use (FIG. 1), the upper shell 2a and the lower shell 2b are joined together with a force, form and/or material fit in a manner known to the person skilled in the art. For example, the upper shell 2a and the lower shell 2b may be screwed, adhesively bonded, welded and/or latched to one another.

The upper part 3 is arranged above the lower part 2 in the height direction Z and comprises a glide face 5 arranged on the upper side. In the embodiment shown, the glide face 5 is formed by two partial glide faces 5a, 5b, which are spaced apart from one another in the transverse direction Y by an upper-side opening (not denoted in detail) on the upper part 3. The glide face 5 is provided for gliding interaction with a femoral component. This femoral component may be formed by a distal end of a femur or a femoral trial implant, which is fixed thereon and has a correspondingly configured glide face. The shaping of the glide face 5 which may be seen with the aid of the figures is modeled in a manner known to the person skilled in the art on the tibial articular face of a femorotibial joint.

In the embodiment shown, the glide face 5 is formed directly on the upper part 3 and to this extent constitutes its upper side. Such a configuration is advantageous but is not to be regarded as compulsory in respect of the present invention. In one embodiment (not shown), the glide face is arranged on a component manufactured separately from the upper part and is firmly connected to the upper part in a state ready for use.

The height adjustment mechanism E is used to adjust an overall height, extending in the height direction Z, of the tibial trial implant 1. In other words, the upper part 3 and therefore the glide face 5 are positioned at different heights above the lower part 2 by means of the height adjustment mechanism E. Such a height adjustment of the glide face 5 is necessary during the knee joint replacement operation for so-called trial repositioning. Trial repositioning is an operation step which precedes the actual knee joint replacement, during which the dimensions and shapes, required for correctly functional replacement of the knee joint, of the tibial and femoral implant components are determined. This use-related background of the tibial trial implant 1 is known to the person skilled in the art. No further explanations are therefore required in this regard.

The height adjustment mechanism E is actively connected in a manner described in more detail below to the upper part 3 and the lower part 2, and allows relative displacement of the upper part 3 in relation to the lower part 2 along the height direction Z. The upper part 3 can thereby be displaced between different adjustment positions in the height direction H, a first adjustment position in which the glide face 5 is positioned at a first height H1 (FIG. 5) above the lower part 2 being shown by way of example with the aid of FIGS. 1 and 5. The first height H1 is indicated by way of example with the aid of FIG. 5 between an upper edge of the glide face 5 and the lower side 4 of the lower part 2. In a second adjustment position (not graphically represented), the upper part 3 is displaced upward in the height direction Z, the glide face 5 being positioned at a second height H2. The second height H2 is schematically indicated in FIG. 5.

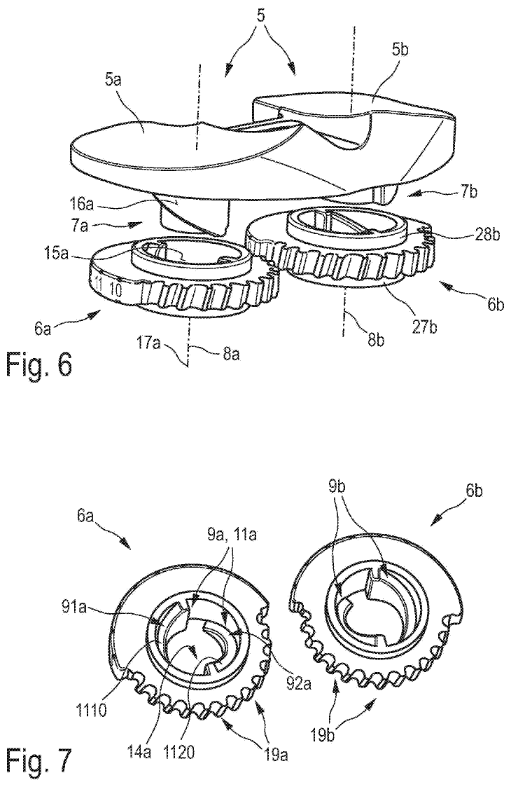

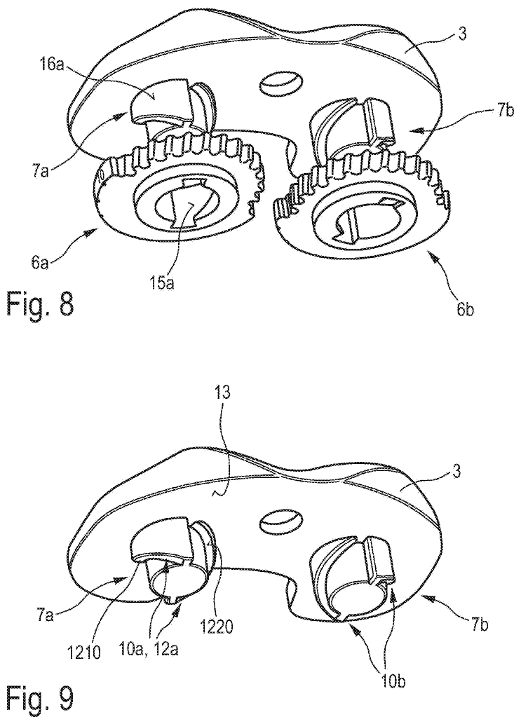

The height adjustment mechanism E comprises a drive wheel 6a and a driven element 7a (cf. FIGS. 6 to 9). The drive wheel 6a is mounted rotatably on the lower part 2 about a first rotation axis 8a oriented parallel to the height direction Z. The drive wheel 6a furthermore comprises a control cam 9a rising in the height direction Z. The driven element 7a is firmly connected to the upper part 3 and comprises a supporting section 10a glidingly supported on the control cam 9a. The drive wheel 6a, its control cam 9a, the driven element 7a and its supporting section 10a form a cam gear. The cam gear makes it possible to transmit rotational movement of the drive wheel 6a about the first rotation axis 8a into a translational movement of the upper part 3 along the height direction Z. In other words, the supporting section 10a glidingly supported on the control cam 9a is pressed upward in the height direction Z during a rotation of the drive wheel 6a. In this way, the upper part 3 can be displaced for the above-described purpose between the first adjustment position and the second adjustment position and the glide face 5 can therefore be positioned between the first height H1 and the second height H2 (FIG. 5). According to terminology which is moreover conventional in the field of gear technology, the drive wheel 6a and the driven element 7a may also be referred to respectively by the terms “cam carrier” and “take-off member”.

In the embodiment shown, the drive wheel 6a is preceded, in a manner described in more detail below, by further component parts and/or sections of the height adjustment mechanism E, which are used for manual and/or tool-driven actuation. Such a configuration is not, however, compulsory. In one embodiment (not shown), the drive wheel itself may be configured for manual actuation and may for this purpose comprise a corresponding actuation section, for example on an outer circumference.

Furthermore, a configuration of the tibial trial implant 1 which is at least substantially, preferably fully, mirror-symmetrical with respect to a mid-length plane X-Z is provided in the embodiment shown. Accordingly, the height adjustment mechanism E is also configured correspondingly mirror-symmetrically with respect to the vertical mid-length plane X-Z. As is shown with the aid of the figures, the height adjustment mechanism E comprises two drive wheels 6a, 6b and two driven elements 7a, 7b. The further drive wheel 6b and the further driven element 7b are arranged spaced apart from the drive wheel 6a and the driven element 7a in the transverse direction Y. The further drive wheel 6b is mounted rotatably on the lower part 2 about a further first rotation axis 8b. The further first rotation axis 8b is oriented parallel to the first rotation axis 8a. The shaping and/or configuration of the further drive wheel 6b and of the further driven element 7a are mirror-symmetrical with the drive wheel 6a and the driven element 7a with respect to the mid-length plane X-Z.

It is explicitly pointed out that the mirror-symmetrical configuration of the present embodiment is advantageous but is not to be regarded as compulsory in respect of the present invention. Accordingly, in one embodiment (not shown), only one drive wheel is provided, which interacts with a driven element.

In order to avoid repetition, reference will primarily be made in detail to the drive wheel 6a and the driven element 7a in the description below. The functional and physical features disclosed in this regard apply, mutatis mutandis, also in respect of the further drive wheel 6b and the further driven element 7b. This also applies vice versa.

The control cam 9a is formed in the present case by a control face 11a, which may also be referred to as an oblique face, helically extending with a constant pitch coaxially around the first rotation axis 8a.

The control cam 9a and therefore also the control face 11a in the present case comprise disjunct subsections, namely a first control cam section 91a and a second control cam section 92a, and respectively a first control face section 1110 and a second control face section 1120. Such a configuration is not, however, compulsory. In one embodiment (not shown), the control cam may be formed by a control face which is integrally continuous along its longitudinal extent.

In principle, the supporting section 10a may be supported on the supporting face 11a pointwise, linearly and/or over a surface. In the present case, support over a surface is provided, so that the supporting section 11a is correspondingly formed by a supporting face 12a. The supporting face 12a is configured complementarily to the control face 11a. The supporting face 12a accordingly extends helically with a constant pitch coaxially around the first rotation axis 8a. Furthermore, a disjunct configuration with two supporting face sections 1210, 1220 is provided.

The driven element 7a in the present case projects downward from a lower side 13 of the upper part 3 along the height direction Z. In this case, the driven element 7a has a pin-like basic shape. The driven element 7a is formed integrally onto the lower side 13 of the upper part 3. In a further embodiment, the driven element may moreover be manufactured separately from the upper part and joined in a manner known per se onto the upper part.

In order to allow the simplest possible replacement of the upper part 3 with a differently configured upper part, which for example comprises a differently configured glide face, in the present case a plug connection is provided between the upper part 3 and the lower part 2. This plug connection is manually releasable in a straightforward way by retracting the upper part 3 upward in the height direction Z from the lower part 2. The plug connection is in this case formed between the drive wheel 6a and the driven element 7a. For this purpose, the drive wheel 6a comprises a cylindrical bore 14a extending coaxially with the first rotation axis 8a. The cylindrical bore 14a is configured in the present case as a through-bore. The cylindrical bore 14a comprises a cylindrical lateral surface 15a. The driven element 7a comprises a plug cylinder complementary to the cylindrical bore 14a, or functions as such a plug cylinder, and is correspondingly provided with a complementary cylindrical lateral surface 16a. The cylindrical lateral surface 16 of the driven element 7a interacts during a rotation actuation of the drive wheel 6a in the circumferential direction about the first rotation axis 8a and axially along the height direction Z in a glidingly mobile fashion with the cylindrical lateral surface 15a of the cylindrical bore 14a.

In the embodiment shown, the control cam 9a is arranged as a kind of radial projection in the cylindrical bore 14a. In one embodiment (not shown), the control cam is instead arranged axially offset in the axial direction with respect to the through-bore 14a on an upper side of the drive wheel 9a. The supporting section 10a is formed by a radial indentation of the cylindrical lateral surface 16a of the plug cylinder. In one embodiment (not shown), the supporting section may be formed at a separate location from the plug cylinder.

The plug cylinder formed by the driven element 7a is inserted top downward in the height direction along the first rotation axis 8a into the cylindrical bore 14a. The first rotation axis 8a therefore coincides in the embodiment shown with a plug axis 17a of the plug connection.

Because of the mirror-symmetrical configuration of the present embodiment, the plug connection between the upper part 3 and the lower part 2 is furthermore formed in the present case between the further drive wheel 6b and the further driven element 7b. In this regard, the comments relating to the plug connection between the drive wheel 6a and the driven element 7a apply correspondingly. Separate reference to the sections required for the movement transmission and formation of the plug connection on the further drive wheel 6b and the further driven element 7b will be omitted for the sake of the required brevity. Instead, reference is made to the relevant explanations in connection with the drive wheel 6a and the driven element 7a.

The height adjustment mechanism E in the present case furthermore comprises a worm gear 18, 19a, 19b preceding the cam gear. The worm gear 18, 19a, 19b comprises a worm drive shaft 18 and an outer circumferential section 19a, toothed complementarily to the worm drive shaft 18, of the drive wheel 6a.

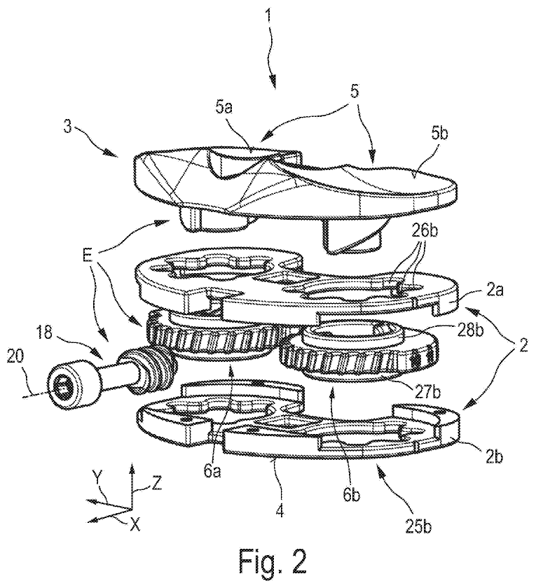

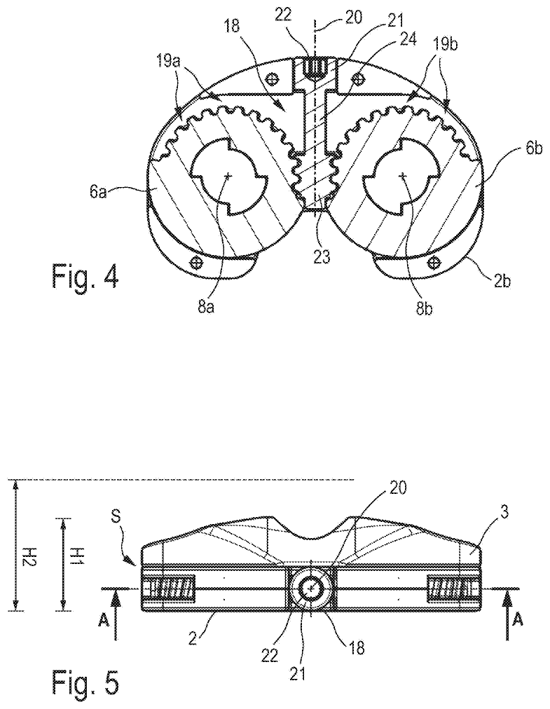

The worm drive shaft 18a is mounted rotatably on the lower part 2 about a second rotation axis 20 (FIG. 2) oriented perpendicularly to the height direction Z and interacts for movement and force transmission with the toothed outer circumferential section 19a (FIG. 4). In the embodiment shown, the worm drive shaft 18 furthermore interacts with a complementarily toothed outer circumferential section 19b of the further drive wheel 6b.

The worm drive shaft 18 projects in the longitudinal direction X between the two drive wheels 6a, 6b. During a rotational movement of the worm drive shaft 18 about the third rotation axis 20, the two drive wheels 6a, 6b are driven counter to one another about the respective first rotation axis 8a, 8b.

The worm gear 18, 19a, 19b formed in this way is configured to be self-retaining. Accordingly, the worm gear 18, 19a, 19b can be driven only by means of a drive-side rotational movement of the worm drive shaft 18. Driving by means of a movement originating from one of the drive wheels 6a, 6b is, conversely, inhibited due to friction and/or transmission and is therefore not possible.

The worm drive shaft 18 comprises a rotation actuation section 21, which in the embodiment shown is provided with a tool face 22. The tool face 22 is a hex socket face, which is configured to interact with a conventional hex key provided for medical use. In one embodiment (not shown), a crank formed by the rotation actuation section, an actuation wheel or the like is provided instead of the tool face 22. The rotation actuation section 21 is arranged at one end of the worm drive shaft 18 and is externally accessible in the assembled state of the tibial trial implant 1 ready for use. At the other end, the worm drive shaft 18 comprises a toothing section 23, which interacts during a rotational movement of the worm drive shaft 18 with the toothed outer circumferential sections 19a, 19b. Between the toothing section 23 and the rotation actuation section 21, a stem section 24 extends in the axial direction.

The drive wheel 6a, the further drive wheel 6b and the worm drive shaft 18 are held between the upper shell 2a and the lower shell 2b of the lower part 2, corresponding glide bearing faces, which for simplified graphical representation are denoted in the figures only in relation to the further drive wheel 6b, being provided in particular for mounting of the drive wheels 6a, 6b. In respect of the glide bearing of the drive wheel 6a, the comments relating to the further drive wheel 6b apply correspondingly. The glide bearing face assigned to the further drive wheel 6b is formed by a through-bore 25b extending coaxially with the further rotation axis 8b through the lower part 2. The through-bore 25b comprises—starting from an imaginary circular-cylindrical shape—a plurality of radial protrusions 26b. The protrusions 26b may also be referred to as flushing protrusions and allow simplified and therefore particularly hygienic flushability of the lower part 2 with a liquid. The further drive wheel 6b comprises a lower radial hub 27b and an upper radial hub 28b, which are provided for gliding contact on the corresponding glide bearing faces of the upper shell 2a and of the lower shell 2b, respectively.

Furthermore, a scale display S formed between the drive wheel 6a and the lower part 2 is provided, the arrangement of which may be seen with the aid of FIG. 5. The scale display S comprises a reading element configured as a reading index, which is arranged both on the upper shell 2a and on the lower shell 2b. Additionally provided is a scale which may be seen in FIG. 6 and has a number sequence. The scale display S allows reading of the adjustment position of the upper part.

Moreover, an embodiment (not graphically represented) is provided in which the trial implant is provided for use in a (general) joint replacement operation and to this extent not necessarily for a knee joint replacement operation. Accordingly, the lower part of this embodiment is provided not necessarily for tibial fixation but in general for bone fixation and/or support. The glide face, arranged on the upper side, of the upper part in this embodiment is provided for gliding interaction with a further component fixed to the bone, which to this extent need not necessarily be a femoral component.

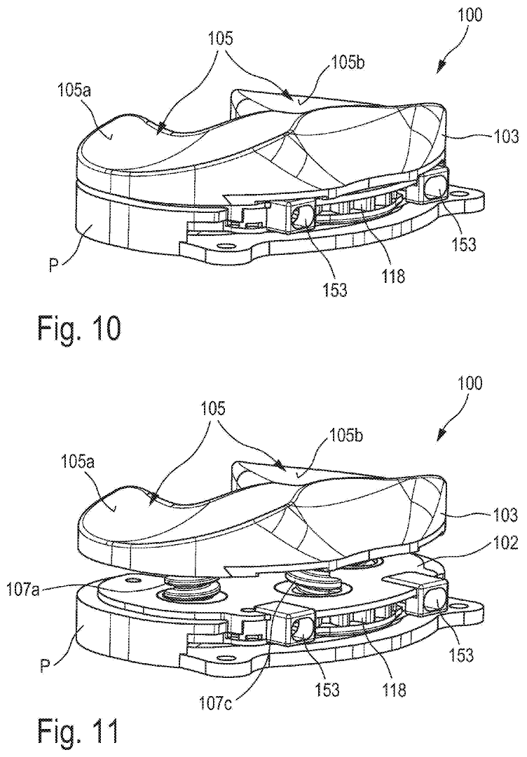

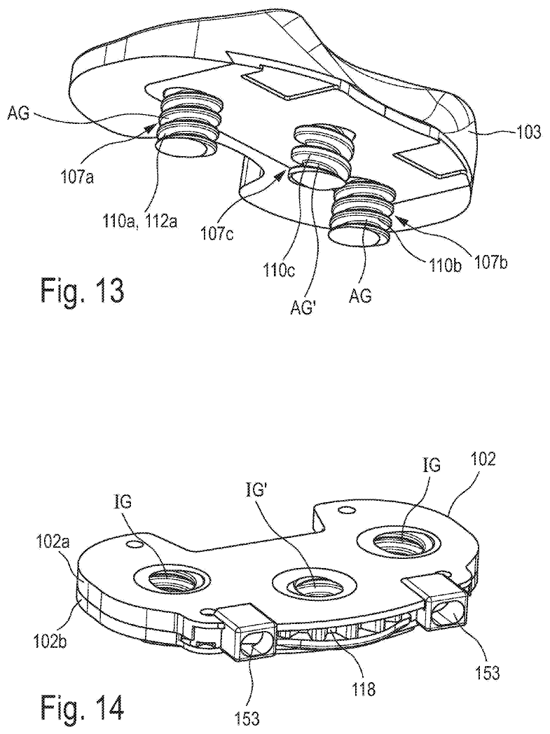

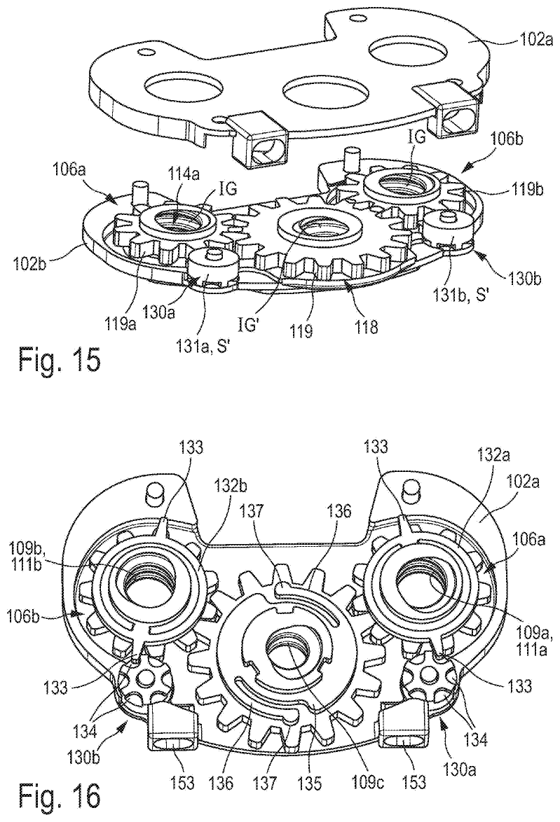

A further embodiment of a tibial trial implant 100 according to the invention is shown with the aid of FIGS. 10 to 18. The basic configuration and functionality of the tibial trial implant 100 largely correspond to the configuration and functionality of the tibial trial implant 1 according to FIGS. 1 to 9. Primarily essential differences of the tibial trial implant 100 will therefore be discussed below. In other regards, in order to avoid repetition, reference is made to the description of the tibial trial implant 1.

The tibial trial implant 100 again comprises a lower part 102, an upper part 103 and a height adjustment mechanism E′.

The lower part 102 (FIG. 14) is in the present case configured for indirect tibial fixation and accordingly interacts with a tibial trial plateau P. In a state ready for use, the lower part 102 is fitted beforehand with its lower side 104 into a reception recess configured therefor (not denoted in detail) of the tibial trial plateau P.

In accordance with the lower part 2, the lower part 102 has a plate-like basic shape and a two-shell configuration with an upper shell 102a and a lower shell 102b.

In accordance with the upper part 3, the upper part 103 comprises a glide face 105 which is arranged on the upper side and has two partial glide faces 105a, 105b spaced apart from one another in the transverse direction Y.

The height adjustment mechanism E′ is—basically in accordance with the height adjustment mechanism E—actively connected on the one hand to the upper part 103 and on the other hand to the lower part 102 and allows relative displacement of the upper part 103 in relation to the lower part 102 along the height direction Z. In this case, FIG. 10 shows by way of example a first adjustment position, which may also be referred to as a lower end location. In the lower end location, the tibial trial implant 100 has its minimum overall height and/or thickness. FIG. 11 shows a second adjustment position, which may also be referred to as an upper end location. In this position, the tibial trial implant 100 assumes its maximum overall height and/or thickness.

The height adjustment mechanism E′ again comprises at least one drive wheel 106a and one driven element 107a. The drive wheel 106a is mounted rotatably on the lower part 102 about a rotation axis (not denoted in detail) oriented parallel to the height direction Z and comprises a control cam 109a rising in the height direction Z. The control cam 109a is formed in the embodiment shown by a control face 111a extending helically with a constant pitch coaxially around said rotation axis.

In the present case, the control cam 109a and/or the control face 111a are formed in the configuration of an internal screw thread IG. The internal screw thread IG is introduced into a cylindrical bore 114a of the drive wheel 106a. The cylindrical bore 114a extends coaxially with the rotation axis of the drive wheel 106a.

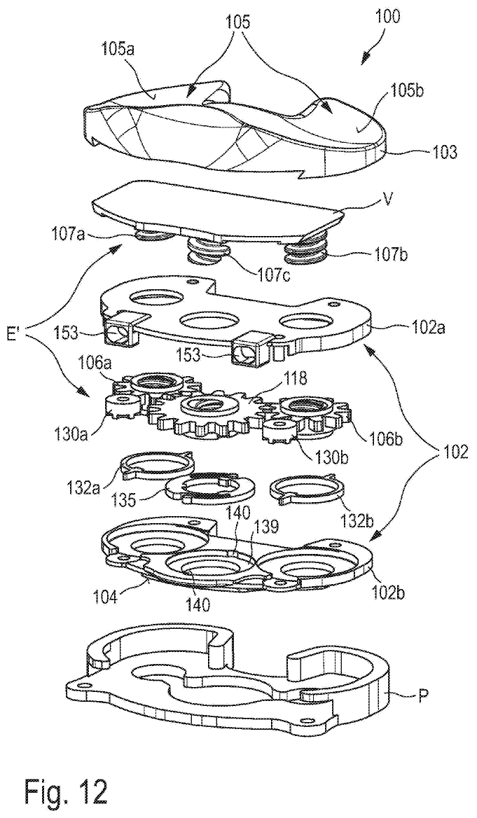

The at least one driven element 107a is firmly connected to the upper part 103. In contrast to the driven element 7a of the tibial trial implant 1, the driven element 107a is not, for instance, formed integrally on the upper part 103. Instead, there is a connecting element V assigned to the upper part 103 (FIG. 12), on which the driven element 107a is formed. The connecting element V is plugged together in a manner known per se to the person skilled in the art releasably in the longitudinal direction and with a form fit in the transverse and height directions Y, Z with the upper part 103. The at least one driven element 107a comprises—basically in accordance with the driven element 7a—a supporting section 110a which is glidingly supported on the control cam 109a. In the embodiment shown, the supporting section 110a is formed by a supporting face 112a extending helically with a constant pitch.

The supporting section 110a and/or the supporting face 112a are configured as an external screw thread AG. The external screw thread AG is complementary to the internal screw thread IG. Expressed simply, the internal screw thread IG and the external screw thread AG form a kind of threaded spindle, by means of which the rotational movement of the drive wheel 106a can be converted into a translational movement of the driven element 107a and therefore also of the upper part 103.

Expressed simply, the driven element 107a may be regarded as a screw and the drive wheel 106a as a nut.

The height adjustment mechanism E′ is—in a similar way to the height adjustment mechanism E—configured at least substantially, preferably fully, mirror-symmetrically. The height adjustment mechanism E′ again comprises at least two drive wheels 106a, 106b and at least two driven elements 107a, 107b. These will also be referred to below as the first drive wheel 106a, second drive wheel 106b, first driven element 107a and second driven element 107b. In respect of the configuration and functionality of the second drive wheel 106b, the comments above relating to the first drive wheel 106a apply correspondingly. Correspondingly, the same applies in respect of the functionality and configuration of the second driven element 107b. In order to avoid repetition, reference is accordingly made to the disclosure relating to the first drive wheel 106a and the first driven element 107a.

The drive wheels 106a, 106b are preceded by a control wheel 118. The control wheel 118 is mounted rotatably on the lower part 102 about a rotation axis oriented parallel to the rotation axes of the drive wheels 106a, 106b. The two drive wheels 106a, 106b and the control wheel 118 respectively comprise spur teeth 119a, 119b, 119. The spur teeth 119 of the control wheel 118 are meshed with the spur teeth 119a of the first drive wheel 106a and with the spur teeth 119b of the second drive wheel 106b. In this way, the respective rotational movements are forcibly coupled and/or synchronized with one another. A rotational movement of the control wheel 118 counterclockwise causes a rotational movement, respectively directed in the clockwise sense, of the first drive wheel 106a and of the second drive wheel 106b.

The spur teeth 119a, 119b of the two drive wheels 106a, 106b are identical in the embodiment shown in respect of their respective number of teeth and the other tooth features. The spur teeth 119 of the control wheel 118 comprise a greater number of teeth than the spur teeth 119a, 119b. An outer diameter (not denoted in detail) of the control wheel 118 is greater than the outer diameter of the drive wheels 106a, 106b.

In the embodiment shown, the control wheel 118 is configured for indirect manual actuation, which will be described in more detail below. In a further embodiment, the control wheel is configured for direct manual rotation actuation.

The control wheel 118 in the present case comprises an internal screw thread IG′ in accordance with the drive wheels 106a, 106b. In correspondence with the internal screw thread IG, the internal screw thread IG′ comprises a control cam (not denoted in detail), or more precisely a control face extending helically with a constant pitch. The internal screw thread IG′ of the control wheel 118 interacts with an external screw thread AG′ of a further driven element 107c. The further driven element 107c will also be referred to below as the third driven element 110c. The external screw thread AG′ of the third driven element 107c again forms a supporting section, or more precisely a control face extending helically with a constant pitch.

The control wheel 118 functions to this extent simultaneously as an additional third drive wheel. The internal screw thread IG′ and the external screw thread AG′ are mutually complementary. The sense of rotation of the internal screw thread IG′ is opposite to the sense of rotation of the internal screw thread IG of the drive wheels 106a, 106b. The same applies correspondingly for the sense of rotation of the external screw thread AG′. Furthermore, the internal screw thread IG′ has a thread pitch which is different to the thread pitch of the internal screw thread IG of the two drive wheels 106a, 106b. Accordingly, the thread pitch of the external screw thread AG′ is also different to the thread pitch of the external screw thread AG of the driven elements 107a, 107b.

The control wheel 118 is arranged centrally between the first drive wheel 106a and the second drive wheel 106b in the transverse direction Y.

The tibial trial implant 100 comprises a first display wheel 130a and a second display wheel 130b. The first display wheel 130a is assigned to the first drive wheel 106a and is coupled thereto so as to transmit movement. The second display wheel 130b is assigned to the second drive wheel 106b and is coupled thereto so as to transmit movement. In a further embodiment, there is only a single display wheel. The configuration and function of the display wheels 130a, 130b are identical, so that only the first display wheel 130a will be discussed below in order to avoid repetition. The disclosure in this regard also applies, mutatis mutandis, for the second display wheel 130b.

The first display wheel 130a is rotationally movably mounted on the lower part 102 and comprises a scale S′ (not graphically represented in detail). The scale S′ is arranged on an outer circumference 131a of the first display wheel 130a. The scale S′ may, for example, be formed by a sequence of graduation lines or numbers. The scale S′ allows reading of the adjustment position of the upper part 103. This is in correspondence with the scale S of the tibial trial implant 1 (FIG. 5).

By the movement-transmitting coupling, a rotational movement of the first drive wheel 106a is converted into a rotational movement of the first display wheel 130a. The movement transmission may be configured to be continuous or intermittent, that is to say stepwise. In the embodiment shown, the latter is the case.

By the intermittent movement transmission explained in more detail below, a continuous rotational movement of the first drive wheel 106a causes a stepwise rotational movement of the display wheel 130a. The intermittent movement transmission is carried out in the present case by means of a first catch element 132a. The second drive wheel 106b and the second display wheel 130b are assigned a second catch element 132b.

The first catch element 132a is connected fixed in rotation to the first drive wheel 106a. In the present case, the first catch element 132a is configured annularly, aligned coaxially with the first drive wheel 106a and fixed with a form, force and/or material fit (not shown in detail) on a lower side of the first drive wheel 106a. The first catch element 132a interacts for movement transmission with a section provided therefor of the display wheel 130a. In the present case, the first catch element 132a comprises at least one, more precisely two, projections 133 protruding outward in the radial direction. These are in the present case arranged offset from one another by 180° in the circumferential direction of the first catch element 132a. The first display wheel 130a comprises indentations 134 set back in the radial direction on its lower side (not denoted in detail) (FIG. 16). For the intermittent movement transmission, the projections 133 engage in the indentations 134.

In the embodiment shown, a (continuous) full revolution of the first drive wheel 106a through 360° causes a stepwise rotational movement of the first display wheel 130a by two increments. Since in the present case there are precisely six indentations 134 arranged offset from one another respectively by 60°, the first display wheel 130a is accordingly moved in rotation respectively by two steps of 60° each.

It is to be understood that the comments above apply correspondingly in respect of the configuration and function of the second catch element 132b and of the second display wheel 130b.

The control wheel 118 is assigned a latch element 135. In a manner described in more detail below, the latch element forms a releasable latch connection with the lower part 102, or more precisely its lower shell 102b. The latch connection can be overridden and is established in defined rotational settings of the control wheel 118 and therefore of the two drive wheels 106a, 106b. In this way, a user receives tactile and/or acoustic feedback during the adjustment of the height adjustment mechanism E′ as soon as a defined rotational setting and therefore height adjustment of the upper part 103 is reached.

In the embodiment shown, the latch element 135 is configured annularly and joined (in a manner not shown) with a force, form and/or material fit onto a lower side of the control wheel 118. In this case, the latch element 135 is aligned coaxially with the control wheel 118 and can be rotated together therewith about its rotation axis. The latch element 135 interacts with a complementary latch counter-element 139, which in the present case is formed as a circular-cylindrical recess, arranged on the lower part (FIG. 12). The circular-cylindrical recess is sunk into an upper side of the lower shell 102b. In the mounted state ready for use, the catch element 135 is received in the circular-cylindrical recess 139. In the embodiment shown, the catch element 135 comprises two spring arms 136 arranged offset by 180°, on the terminal ends of which a latch projection 137 is respectively formed. The latch projections 137 are resiliently mobile in the radial direction and interact with latch recesses 140 for the latching.

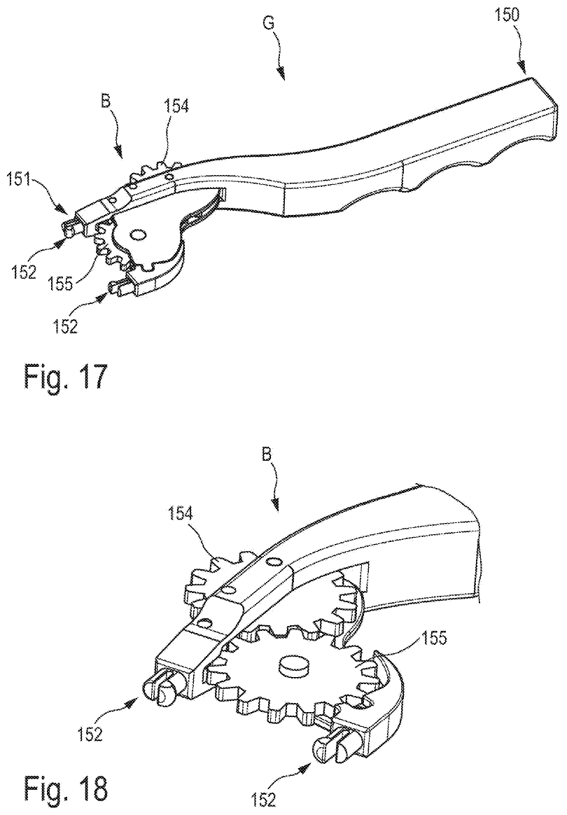

The tibial trial implant 100 furthermore comprises a handle G shown with the aid of FIGS. 17 and 18, which can be connected releasably (in a manner not described in detail) with the lower part 102. The handle G is used on the one hand for simplified handling of the lower part 102. On the other hand, the handle G comprises actuation mechanics B, which are used for simplified and particularly ergonomic actuation of the height adjustment mechanism E′.

The handle G extends longitudinally between a proximal end 150 and a distal end 151. The actuation mechanics B are in the present case arranged in the region of the distal end 151. For the releasable connection to the lower part 102, the handle G in the present case comprises two connecting elements, respectively in the form of a latch element 152, arranged at the distal end 151. In one embodiment (not shown), there is only one latch element. The two latch elements 152 are arranged spaced apart from one another in the transverse direction Y and, in a state connected to the lower part 102, interact respectively with a complementary connecting element in the form of a latch counter-element 153. The latch counter-elements 153 are accordingly arranged spaced apart from one another in the transverse direction Y and in the present case are furthermore positioned on either side of the control wheel 118.

In the embodiment shown, the latch elements 152 are respectively configured as a male latch element and the latch counter-elements 153 are respectively configured as a female latch element, or as a latch slot. The latch elements 152 protrude in the distal direction from the distal end 151 of the handle G. The complementary latch elements 153 extend in the distal direction of the handle G into the lower part 102. The latch counter-elements 153 are formed on the upper shell 102a.

The actuation mechanics B comprise an actuation wheel 154, which, in a state of the handle G connected to the lower part 102, is actively connected at least indirectly to the drive wheels 106a, 106b so as to transmit forces and movement. The actuation wheel 154 is mounted rotatably on the handle G about a rotation axis (not denoted in detail) and may also be referred to as a thumbwheel. A rotation of the actuation wheel 154 causes a rotation of the drive wheels 106a, 106b and therefore a corresponding height adjustment of the upper part 103.

In the embodiment shown, the actuation wheel is actively connected indirectly by means of a transmission wheel 155, mounted rotatably on the handle G, and the control wheel 118 to the drive wheels 106a, 106b. For this purpose, the actuation wheel 154 is provided with spur teeth (not denoted in detail) which are engaged with spur teeth of the transmission wheel 155. The spur teeth of the transmission wheel 155 are in turn meshed with the spur teeth 119 of the control wheel 118. When the handle G is latched with the lower part 102, the teeth of the transmission wheel 155 engage with the teeth 119 of the control wheel 118. When the handle G is retracted from the lower part 102, the meshing between the transmission wheel 155 and the control wheel 118 is released.

1.-20. (canceled)

21. A tibial trial implant for use in a knee joint replacement operation, comprising:
a lower part, which is provided for tibial fixation;
an upper part, which is arranged above the lower part in the height direction and has a glide face that is arranged on the upper side for gliding interaction with a femoral component; and
a height adjustment mechanism, which is actively connected to the upper part and the lower part and by which the upper part is displaceably guidable relative to the lower part in the height direction between:
a first adjustment position, in which the glide face is positioned at a first height above the lower part, and
a second adjustment position, in which the glide face is positioned at a second height above the lower part,
wherein the height adjustment mechanism comprises a cam gear having at least one drive wheel, which is mounted rotatably on the lower part about a first rotation axis oriented parallel to the height direction and comprises a control cam rising in the height direction, and having at least one driven element, which is firmly connected to the upper part and comprises a supporting section glidingly supported on the control cam, so that the upper part is displaceable by rotational movement of the drive wheel between the first adjustment position and the second adjustment position, and
wherein the upper part and the lower part are releasably plugged together by a plug connection formed between the at least one drive wheel and the at least one driven element while being glidingly mobile relative to one another along a plug axis, the plug axis being oriented coaxially with the first rotation axis.

22. The tibial trial implant according to claim 21, wherein the control cam is formed by a control face helically extending with a constant pitch coaxially around the first rotation axis.

23. The tibial trial implant according to claim 21, wherein the supporting section is formed by a supporting face helically extending with a constant pitch coaxially around the first rotation axis.

24. The tibial trial implant according to claim 21, wherein the at least one drive wheel comprises a cylindrical bore extending coaxially with the first rotation axis, into which a complementary plug cylinder of the at least one driven element is releasably plugged so as to form the plug connection.

25. The tibial trial implant according to claim 21, wherein the height adjustment mechanism comprises a worm gear preceding the cam gear.

26. The tibial trial implant according to claim 25, wherein the worm gear comprises self-retention.

27. The tibial trial implant according to claim 25, wherein the worm gear comprises a worm drive shaft, which is mounted rotatably on the lower part about a second rotation axis oriented perpendicularly to the height direction, the worm drive shaft interacting with a toothed outer circumferential section of the at least one drive wheel or with a worm wheel preceding the at least one drive wheel.

28. The tibial trial implant according to claim 27, wherein the worm drive shaft comprises on one end a rotation actuation section configured for manual and/or tool-driven rotation actuation of the worm drive shaft about the second rotation axis.

29. The tibial trial implant according to claim 27, wherein the worm drive shaft is glidingly and rotatably mounted on a glide bearing face of the lower part, the glide bearing face comprising a plurality of radial flushing protrusions.

30. The tibial trial implant according to claim 27, wherein the lower part is configured as two shells and comprises an upper shell and a lower shell, the worm drive shaft being held between the upper shell and the lower shell.

31. The tibial trial implant according to claim 21, wherein the at least one drive wheel is glidingly and rotatably mounted on a glide bearing face of the lower part, the glide bearing face comprising a plurality of radial flushing protrusions.

32. The tibial trial implant according to claim 21, wherein the lower part is configured as two shells and comprises an upper shell and a lower shell, the at least one drive wheel being held between the upper shell and the lower shell.

33. The tibial trial implant according to claim 21,
wherein the height adjustment mechanism is configured at least substantially mirror-symmetrically with respect to a vertical mid-length plane, and
wherein the at least one drive wheel comprises at least two drive wheels,
the tibial trial implant further comprising at least two driven elements.

34. The tibial trial implant according to claim 21, wherein the at least one drive wheel comprises at least two drive wheels and the tibial trial implant further comprises at least one control wheel, the at least two drive wheels and the at least one control wheel respectively comprising spur teeth, with the at least one control wheel being spur-meshed with the at least two drive wheels.

35. The tibial trial implant according to claim 34, wherein the at least one control wheel comprises a control cam that interacts with a further supporting section of a further driven element.

36. The tibial trial implant according to claim 21, further comprising a latch device comprising a latch element actively connected and fixed in rotation to the at least one drive wheel and a complementary latch counter-element arranged on the lower part, the latch element and the latch counter-element interacting in defined rotational settings of the at least one drive wheel while forming an overridable latch connection.

37. The tibial trial implant according to claim 21, further comprising a handle releasably connectable to the lower part and having actuation mechanics, the actuation mechanics comprising an actuation wheel which is actively connected to the at least one drive wheel when the handle is connected to the lower part.

38. The tibial trial implant according to claim 37, wherein the handle comprises at least one connecting element arranged at its distal end, which is releasably connected to a complementary connecting element of the lower part in order to connect the handle to the lower part.

39. A tibial trial implant for use in a knee joint replacement operation, comprising:
a lower part, which is provided for tibial fixation;
an upper part, which is arranged above the lower part in a height direction and has a glide face that is arranged on the upper side and is provided for gliding interaction with a femoral component; and
a height adjustment mechanism, which is actively connected to the upper part and the lower part and by which the upper part is displaceably guidable relative to the lower part in the height direction between:
a first adjustment position, in which the glide face is positioned at a first height above the lower part, and
a second adjustment position, in which the glide face is positioned at a second height above the lower part,
wherein the height adjustment mechanism comprises a cam gear having at least one drive wheel that is mounted rotatably on the lower part about a first rotation axis oriented parallel to the height direction and comprises a control cam rising in the height direction, and having at least one driven element, which is firmly connected to the upper part and comprises a supporting section glidingly supported on the control cam, so that the upper part is displaceable by a rotational movement of the drive wheel between the first adjustment position and the second adjustment position,
wherein a scale display is formed between the lower part and the at least one drive wheel, which comprises a reading element and a scale and allows reading of the adjustment position of the upper part and/or of the height of the glide face.

40. The tibial trial implant according to claim 39, wherein the control cam is formed by a control face helically extending with a constant pitch coaxially around the first rotation axis.

41. The tibial trial implant according to claim 39, wherein the supporting section is formed by a supporting face helically extending with a constant pitch coaxially around the first rotation axis.

42. The tibial trial implant according to claim 39, wherein the at least one drive wheel comprises a cylindrical bore extending coaxially with the first rotation axis, into which a complementary plug cylinder of the at least one driven element is releasably plugged so as to form the plug connection.

43. The tibial trial implant according to claim 39, wherein the height adjustment mechanism comprises a worm gear preceding the cam gear.

44. The tibial trial implant according to claim 39, wherein the worm gear comprises self-retention.

45. The tibial trial implant according to claim 39, wherein the worm gear comprises a worm drive shaft, which is mounted rotatably on the lower part about a second rotation axis oriented perpendicularly to the height direction, the worm drive shaft interacting with a toothed outer circumferential section of the at least one drive wheel or with a worm wheel preceding the at least one drive wheel.

46. The tibial trial implant according to claim 45, wherein the lower part is configured as two shells and comprises an upper shell and a lower shell, the worm drive shaft being held between the upper shell and the lower shell.

47. The tibial trial implant according to claim 45, wherein the worm drive shaft comprises on one end a rotation actuation section configured for manual and/or tool-driven rotation actuation of the worm drive shaft about the second rotation axis.

48. The tibial trial implant according to claim 39, wherein the lower part is configured as two shells and comprises an upper shell and a lower shell, the at least one drive wheel being held between the upper shell and the lower shell.

49. The tibial trial implant according to claim 39,
wherein the height adjustment mechanism is configured at least substantially mirror-symmetrically with respect to a vertical mid-length plane, and
wherein the at least one drive wheel comprises at least two drive wheels,
the tibial trial implant further comprising at least two driven elements.

50. A tibial trial implant for use in a knee joint replacement operation, comprising:
a lower part, which is provided for tibial fixation;
an upper part, which is arranged above the lower part in the height direction and has a glide face that is arranged on the upper side and is provided for gliding interaction with a femoral component; and
a height adjustment mechanism, which is actively connected to the upper part and the lower part and by which the upper part can be guided displaceably relative to the lower part in the height direction between:
a first adjustment position, in which the glide face is positioned at a first height above the lower part, and
a second adjustment position, in which the glide face is positioned at a second height above the lower part,
wherein the height adjustment mechanism comprises a cam gear having at least one drive wheel that is mounted rotatably on the lower part about a first rotation axis oriented parallel to the height direction and comprises a control cam rising in the height direction, and having at least one driven element, which is firmly connected to the upper part and comprises a supporting section glidingly supported on the control cam, so that the upper part is displaceable by a rotational movement of the drive wheel between the first adjustment position and the second adjustment position,
wherein the at least one drive wheel is coupled so as to transmit movement to at least one display wheel that is rotationally mounted on the lower part, the display wheel comprising a scale that allows reading of the adjustment position of the upper part and/or of the height of the glide face.

51. The tibial trial implant according to claim 50, wherein the control cam is formed by a control face helically extending with a constant pitch coaxially around the first rotation axis.

52. The tibial trial implant according to claim 50, wherein the supporting section is formed by a supporting face helically extending with a constant pitch coaxially around the first rotation axis.

53. The tibial trial implant according to claim 50, wherein the at least one drive wheel comprises a cylindrical bore extending coaxially with the first rotation axis, into which a complementary plug cylinder of the at least one driven element is releasably plugged so as to form the plug connection.

54. The tibial trial implant according to claim 50, wherein the lower part is configured as two shells and comprises an upper shell and a lower shell, the at least one drive wheel being held between the upper shell and the lower shell.

55. The tibial trial implant according to claim 50,
wherein the height adjustment mechanism is configured at least substantially mirror-symmetrically with respect to a vertical mid-length plane, and
wherein the at least one drive wheel comprises at least two drive wheels,
the tibial trial implant further comprising at least two driven elements.

56. The tibial trial implant according to claim 50, wherein the at least one drive wheel comprises at least two drive wheels, and the tibial trial implant further comprises at least one control wheel, the at least two drive wheels and the at least one control wheel respectively comprising spur teeth, with the at least one control wheel being spur-meshed with the at least two drive wheels.

57. The tibial trial implant according to claim 56, wherein the at least one control wheel comprises a further control cam, which interacts with a further supporting section of a further driven element.

58. The tibial trial implant according to claim 50, further comprising at least one catch element connected and fixed in rotation to the at least one drive wheel, which intermittently interacts with the at least one display wheel so that a continuous rotational movement of the at least one drive wheel causes a stepwise rotational movement of the at least one display wheel.

59. The tibial trial implant according to claim 50, further comprising a latch device comprising a latch element actively connected and fixed in rotation to the at least one drive wheel and a complementary latch counter-element arranged on the lower part, the latch element and the latch counter-element interacting in defined rotational settings of the at least one drive wheel while forming an overridable latch connection.

60. The tibial trial implant according to claim 50, further comprising a handle releasably connectable to the lower part and having actuation mechanics, the actuation mechanics comprising an actuation wheel that is actively connected to the at least one drive wheel when the handle is connected to the lower part.

61. The tibial trial implant according to claim 60, wherein the handle comprises a distal end and at least one connecting element arranged at the distal end, the at least one connecting element being releasably connected to a complementary connecting element of the lower part to connect the handle to the lower part.
